(19) United States
(12) Patent Application Publication
(10) Pub. No.: US 2023/0260607 A1
(43) Pub. Date: Aug. 17, 2023

(54) INFORMATION PROCESSING APPARATUS, INFORMATION PROCESSING METHOD AND PROGRAM

(71) Applicant: NIPPON TELEGRAPH AND TELEPHONE CORPORATION, Tokyo (JP)

(72) Inventors: Erika ASHIKAGA, Tokyo (JP); Mizue HAYASHI, Tokyo (JP); Atsunobu KIMURA, Tokyo (JP)

(21) Appl. No.: 18/006,175

(22) PCT Filed: Jul. 27, 2020
(86) PCT No.: PCT/JP2020/028741

§ 371 Date: Jan. 20, 2023

(51) Int. Cl. G16H 10/60; G06N 5/02

(52) U.S. Cl. CPC G16H 10/60; G06N 5/02

(57) ABSTRACT

The information processing apparatus is capable of efficiently acquiring expert knowledge by including: a classification unit that classifies a plurality of pieces of data including pieces of personal feature information, each of which is a piece of information indicating a feature of an individual, into groups on the basis of similarity of the pieces of information indicating the features; a presentation unit that presents one piece of data representing data included in the groups; an input unit that receives input of information that implicitly indicates a feature indicated by the personal feature information included in the data presented by the presentation unit; and an update unit that records the data whose input of the information is received by the input unit in a storage unit in association with the information received by the input unit.

### TECHNICAL FIELD

The present invention relates to an information processing apparatus, an information processing method, and a program.

### BACKGROUND ART

A care record contains daily physical information about a care receiver, as well as the state of the care receiver and utterances of the care receiver.

Conventionally, there has been a technique for enabling leveling of the described contents of the care records by analyzing the care records so as to allow for seamless sharing of the information of the care receivers between the caregivers and by referring to the recorded contents of experienced caregivers.

### CITATION LIST

### Non Patent Literature

[Non-patent literature 1] “Analysis of care recorded contents using TETDM”, Kushima, Araki, Suzuki, Yamasaki, Sonehara, Japan Association of Medical Informatics, Spring Conference Paper, 35 (5) 229-238, 2015.

### SUMMARY OF INVENTION

### Technical Problem

Since the information described in a care record (the physical information of a care receiver, the condition of the care receiver, utterances of the care receiver, and the like) are explicit information that can be relatively easily grasped from the outside, it may be difficult to solve the essential problems only by means of care based on such explicit information.

For example, for a person who complains that he/she has trouble sleeping due to a light mental disorder and a strong sense of loneliness and anxiety, the contents of this claim amount to the explicit information. Based on such explicit information, it is considered that the prescription of a sleeping medicine is an effective countermeasure against elimination of insomnia. However, in essence, the person in question needs someone who could distract him/her from his/her loneliness and listen to him/her, and there have been cases where the person in question was introduced to such a person, had his/her various thoughts and feelings heard and empathized completely, thereby creating a place for him/her and improving his/her symptoms. For a day service user (an elderly person requiring care) whose physical information indicates a decline in muscle strength, such information amounts to the explicit information. Based on such explicit information, it is considered that execution of rehabilitation in a facility is an effective countermeasure. However, in essence, measures need to be taken in consideration of the thoughts and wishes of the person such as things that matter to him/her. For example, if the person “likes cars,” an opportunity to work for a company in the city (e.g., washing cars at a dealer) is provided as a part of rehabilitation in consideration of the fact that he/she “likes cars.” In some cases, these efforts have resulted in the implementation of rehabilitation to cope with muscle weakness through the realization of a life with social connections and a sense of fulfillment. In order to provide effective care to achieve the examples described above, it is considered important to understand information implied in explicit information (e.g., the relationship between the care receiver and people around him/her, the personality, ideas and the like of the care receiver that are not explicitly put into words, which is referred to as “implicit information” hereinafter).

However, in order to derive implicit information from explicit information, specialized knowledge possessed by experts is required, and obtaining expert knowledge is expensive.

The present invention has been made in view of the above-described problems, and an object thereof is to enable the efficient acquisition of expert knowledge.

### Solution to Problem

In order to solve the foregoing problems, an information processing apparatus includes: a classification unit that classifies a plurality of pieces of data including pieces of personal feature information, each of which is a piece of information indicating a feature of an individual, into groups on the basis of similarity of the pieces of information indicating the features; a presentation unit that presents one piece of data representing data included in the groups; an input unit that receives input of information that implicitly indicates a feature indicated by the personal feature information included in the data presented by the presentation unit; and an update unit that records the data whose input of the information is received by the input unit in a storage unit in association with the information received by the input unit.

### Advantageous Effects of Invention

Efficient acquisition of expert knowledge is possible.

### BRIEF DESCRIPTION OF DRAWINGS

FIG. 1 is a diagram illustrating an example of a hardware configuration of an information processing apparatus 10 according to a first embodiment.

FIG. 2 is a diagram illustrating an example of a functional configuration of the information processing apparatus 10 according to the first embodiment.

FIG. 3 is a flowchart for describing an example of a processing procedure executed by an extraction unit 11 according to the first embodiment.

FIG. 4 is a diagram illustrating extraction rules for basic information and extraction examples for the basic information.

FIG. 5 is a diagram illustrating extraction rules for abilities/experiences and extraction examples for the abilities/experiences.

FIG. 6 is a diagram illustrating an example of dictionary information of nouns related to occupations.

FIG. 7 is a diagram illustrating extraction rules for hobbies/preferences and extraction examples for the hobbies/preferences.

FIG. 8 is a diagram illustrating extraction rules for relationships and extraction examples for relationships.

FIG. 9 is a diagram illustrating an example of dictionary information of nouns related to an area.

FIG. 10 is a flowchart for describing an example of a processing procedure executed by an acquisition unit 12 according to the first embodiment.

FIG. 11 is a diagram illustrating an example of a configuration of an expert DB123.

FIG. 12 is a diagram illustrating an example of a configuration of the expert DB123.

FIG. 13 is a diagram illustrating an example of a configuration of the expert DB123.

FIG. 14 is a diagram illustrating an example of a configuration of the expert DB123.

FIG. 15 is a diagram illustrating an example of a configuration of the expert DB123.

FIG. 16 is a diagram illustrating an example of a configuration of the expert DB123.

FIG. 17 is a diagram illustrating an example of a configuration of the expert DB123.

FIG. 18 is a diagram illustrating an example of a configuration of a person-in-question narrative table.

FIG. 19 is a diagram illustrating an example of a configuration of a person-in-question narrative table.

FIG. 20 is a diagram illustrating an example of a configuration of a person-in-question narrative table.

FIG. 20 is a diagram illustrating an example of a configuration of a person-in-question narrative table.

FIG. 22 is a diagram illustrating an example of a functional configuration of the information processing apparatus 10 according to a second embodiment.

FIG. 23 is a flowchart for describing an example of a processing procedure executed by an imparting unit 13 according to the second embodiment.

FIG. 24 is a diagram for explaining an example of calculating an importance level in consideration of a past person-in-question narrative table.

FIG. 25 is a diagram illustrating a configuration example of a person-in-question narrative table to which an importance level is imparted.

FIG. 26 is a diagram illustrating a configuration example of a person-in-question narrative table to which an importance level is imparted.

FIG. 26 is a diagram illustrating a configuration example of a person-in-question narrative table to which an importance level is imparted.

FIG. 26 is a diagram illustrating a configuration example of a person-in-question narrative table to which an importance level is imparted.

FIG. 29 is a diagram illustrating an example of a functional configuration of the information processing apparatus 10 according to a third embodiment.

FIG. 30 is a flowchart for describing an example of a processing procedure executed by the information processing apparatus 10 according to the third embodiment.

FIG. 31 is a diagram illustrating an example of assigning an ID of representative data to non-representative data.

FIG. 32 is a diagram illustrating an example of accepting input for “expert knowledge” of representative data.

FIG. 33 is a diagram illustrating an example of copying “expert knowledge” to non-representative data.

FIG. 34 is a diagram illustrating a first example of expert data added to the expert DB123.

FIG. 35 is a diagram illustrating a second example of expert data added to the expert DB123.

### DESCRIPTION OF EMBODIMENTS

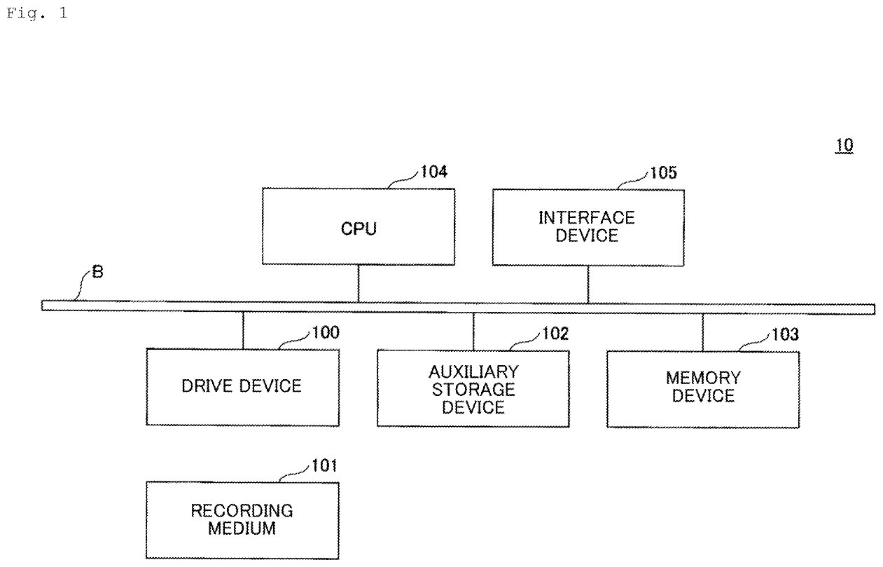

Embodiments of the present invention will be described hereinafter with reference to the drawings. FIG. 1 is a diagram illustrating an example of a hardware configuration of an image processing apparatus 10 according to a first embodiment. The information processing apparatus 10 in FIG. 1 includes a drive device 100, an auxiliary storage device 102, a memory device 103, a CPU 104, an interface device 105, and the like, which are connected to each other via a bus B.

A program that achieves processing in the information processing apparatus 10 is provided by a recoding medium 101 such as a CD-ROM. When the recoding medium 101 that stores the program is set in the drive device 100, the program is installed in the auxiliary storage device 102 from the recoding medium 101 via the drive device 100. However, the program does not necessarily have to be installed from the recoding medium 101 and may be downloaded from another computer via a network. The auxiliary storage device 102 stores the installed program as well as necessary files and data.

The memory device 103 reads the program from the auxiliary storage device 102 and stores the program when an instruction for starting the program is issued. The CPU 104 executes functions relevant to the information processing apparatus 10 according to the program stored in the memory device 103. The interface device 105 is used as an interface for connecting to a network.

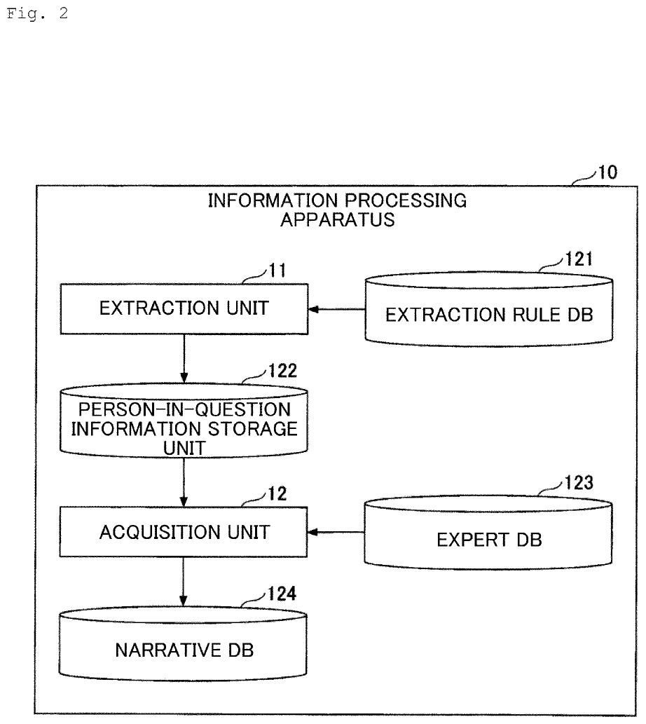

FIG. 2 is a diagram illustrating an example of a functional configuration of the information processing apparatus 10 according to the first embodiment. In FIG. 2, the information processing apparatus 10 includes an extraction unit 11, an acquisition unit 12, and the like. Each of the units is realized by processing of causing the CPU 104 to execute one or more programs installed in the information processing apparatus 10. The information processing apparatus 10 also uses storage units such as a rule DB 121, a person-in-question information storage unit 122, an expert DB123, and a narrative DB124. These storage units can be achieved by using, for example, the auxiliary storage device 102, a storage device that can be connected to the information processing apparatus 10 via a network, or the like.

The information processing apparatus 10 uses the functional configuration illustrated in FIG. 2 to execute processing for acquiring and outputting information implied by personal feature information indicating a feature of a certain person (care receiver) (such as acts and utterances of the certain person, explicit information such as physical information of a target person, etc., which are referred to as “person-in-question information” hereinafter) extracted from information recorded for the certain person (hereinafter referred to as “recorded information”). In the present embodiment, the information implied by information indicating a feature can be obtained from knowledge of an expert of care, as described later.

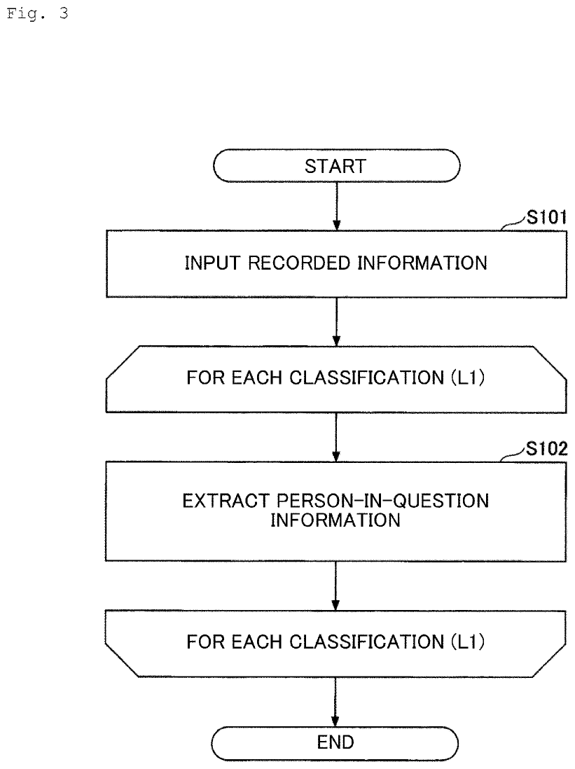

A processing procedure executed by the information processing apparatus 10 will be described hereinafter. FIG. 3 is a flowchart for describing an example of a processing procedure executed by the extraction unit 11 according to the first embodiment.

In step S101, the extraction unit 11 inputs recorded information related to a person such as a care receiver who is a knowledge acquisition target person (referred to as “target person” hereinafter). The recorded information refers to physical information of the target person and explicit recorded information about the state, acts, and utterances of the target person. For example, text data of a care record (date report) of the target person, text data of a conference minute related to the target person, text data which is a voice recognition result of voice data on a conversation between the target person and the caregiver in care, and the like may be input as the recorded information. Here, the conference is, for example, a meeting between the caregiver, the target person and a family member of the target person to discuss plans regarding the future care of the target person. When the processing procedure subsequent to FIG. 3 is periodically executed at intervals of a cycle T1 for each care receiver, the recorded information input in step S101 is the recorded information recorded in the latest cycle T1.

Subsequently, the extraction unit 11 extracts, for each classification (category) defined in advance with respect to the person-in-question information, the person-in-question information on the classification from the recorded information (S102). In the present embodiment, the person-in-question information is classified into “basic information,” “ability/experience,” “hobby/preference,” “relationship,” and the like. These classifications are separated based on the classification of knowledge in expert data stored in an expert DB123 to be described later.

Extraction of person-in-question information related to each classification is performed based on an extraction rule defined for each classification. The extraction rule for each classification is stored in a rule DB121. However, the extraction rule may be incorporated in the extraction unit 11 as a program logic.

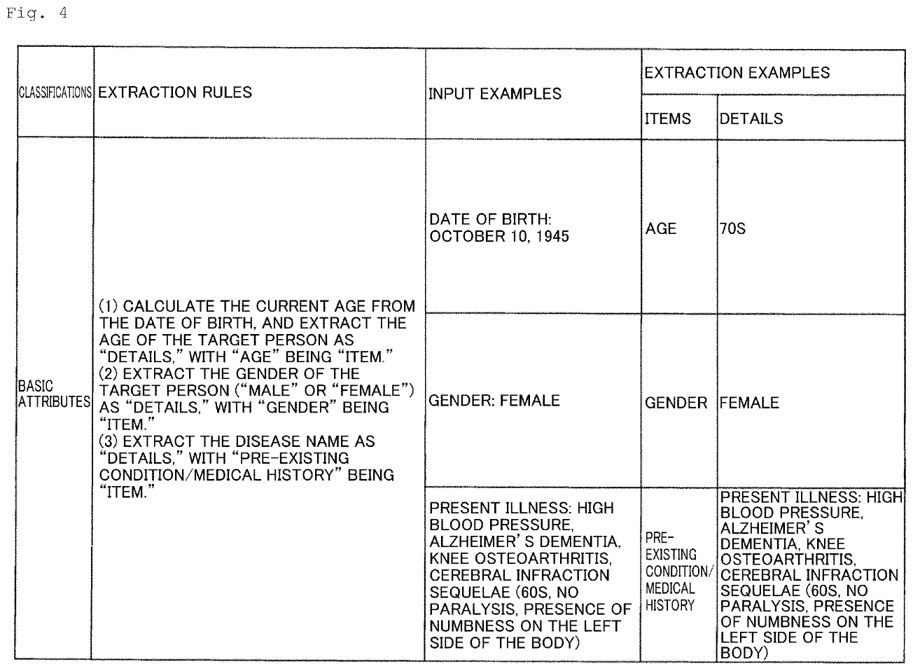

FIG. 4 is a diagram illustrating extraction rules for basic information and extraction examples for the basic information. As shown in FIG. 4, for the basic information, three extraction rules (1) to (3) are defined.

The rule (1) is an extraction rule for calculating the current age from the date of birth, extracting the age of the target person as “details,” with “age” being “item.”

The rule (2) is an extraction rule for extracting the gender of the target person (“male” of “female”) as “details,” with “gender” being “item.”

The rule (3) is an extraction rule for extracting a disease name as “details,” with “pre-existing condition/medical history” being “item.”

In FIG. 4, the column for “input example” is, for example, an example of a part of a text included in the recorded information that can be applied to the basic information extraction rule. The column for “extraction example” is an example of the person-in-question information extracted as a result of applying an extraction rule to “input example.” As for the basic information, as is clear from the extraction rules (1) to (3), items of “age,” “gender,” and “pre-existing condition/medical history” and “details” for each item are extracted as the person-in-question information.

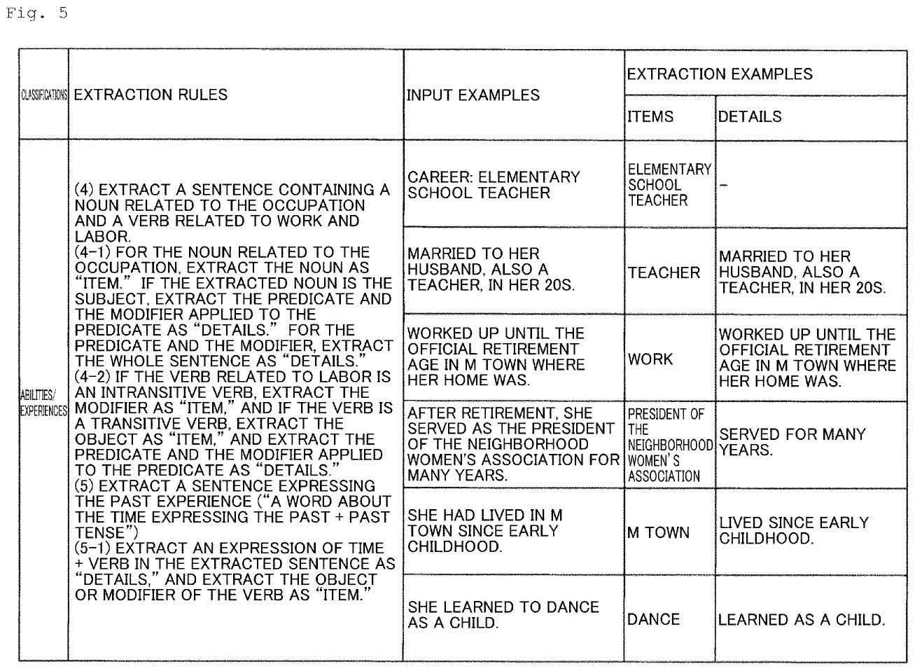

FIG. 5 is a diagram illustrating extraction rules for abilities/experiences and extraction examples for the abilities/experiences. As shown in FIG. 5, as for abilities/experiences, a rule (4-1) and rule (4-2) based on a rule (4) and a rule (5-1) based on a rule (5) are defined.

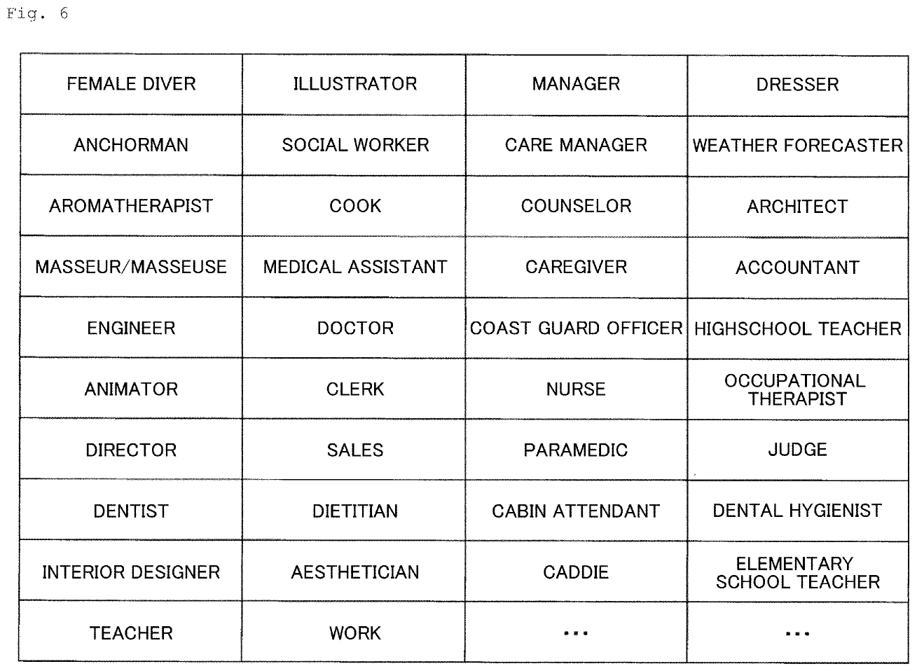

The rule (4) is an extraction rule for extracting a sentence including a noun related to an occupation and a verb related to work and labor. Note that the noun related to an occupation may be determined by, for example, referring to dictionary information shown in FIG. 6. The dictionary information shown in FIG. 6 includes a set of nouns related to occupations. The dictionary information may similarly be prepared for verbs related to work and labor, and the verbs may be determined based on the dictionary information. Not only the nouns that exactly match the dictionary information, but also nouns containing such nouns or nouns containing such nouns at the end may be extracted as the corresponding nouns.

The rule (4-1) is an extraction rule for extracting the noun as “item” when said noun relates to an occupation, extracting the predicate and the modifier applied to the predicate as “details” when the extracted noun is included in the subject, and extracting the whole sentence as “details” when the extracted noun is included in the predicate or modifier.

The rule (4-2) is an extraction rule for extracting a modifier when the verb related to work is an intransitive verb, extracting an object as “item” when the verb is a transitive verb, and extracting a predicate and a modifier applied to the predicate as “details.”

The rule (5) is an extraction rule for extracting a sentence expressing a past experience. For example, a sentence having a format such as “a word about the time expressing the past+past tense” may be extracted as said sentence.

The rule (5-1) is an extraction rule for extracting an expression of time+verb as “details” in the sentence extracted on the basis of the rule (5), and extracting an object or modifier of the verb as “item.”

In FIG. 5, the meanings of the respective columns for “input example” and “extraction example” are the same as those in FIG. 4.

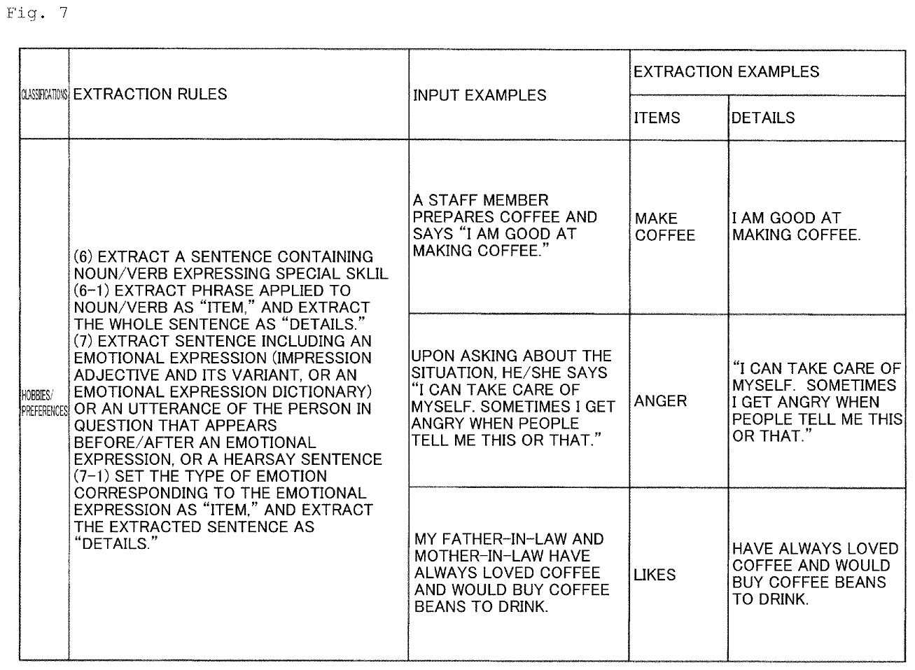

FIG. 7 is a diagram illustrating extraction rules for hobbies/preferences and extraction examples for the hobbies/preferences. As for a hobby and a preference, there are a rule (6-1) based on a rule (6) and a rule (7-1) based on a rule (7).

The rule (6) is an extraction rule for extracting a sentence including a noun or a verb expressing a special skill. The dictionary information may also be prepared in advance for nouns or verbs expressing special skills, and such nouns and verbs may be determined based on the dictionary information.

The rule (6-1) is an extraction rule for extracting a noun or a verb in the sentence extracted based on the rule (6) as “item” and extracting the whole sentence as “details.”

The rule (7) is an extraction rule for extracting a sentence including an emotional expression, an utterance of the person in question that appears before/after an emotional expression, or a hearsay sentence. The emotional expression may be determined by an impression adjective and its variant, or may be determined by referring to an emotional expression dictionary (e.g., “http://www.jnlp.org/SNOW/D18”.)

The rule (7-1) is an extraction rule for extracting the extracted sentence as “details,” with the type of emotion corresponding to an emotional expression being “item.”

In FIG. 7, the meanings of the respective columns for “input example” and “extraction example” are the same as those in FIG. 4.

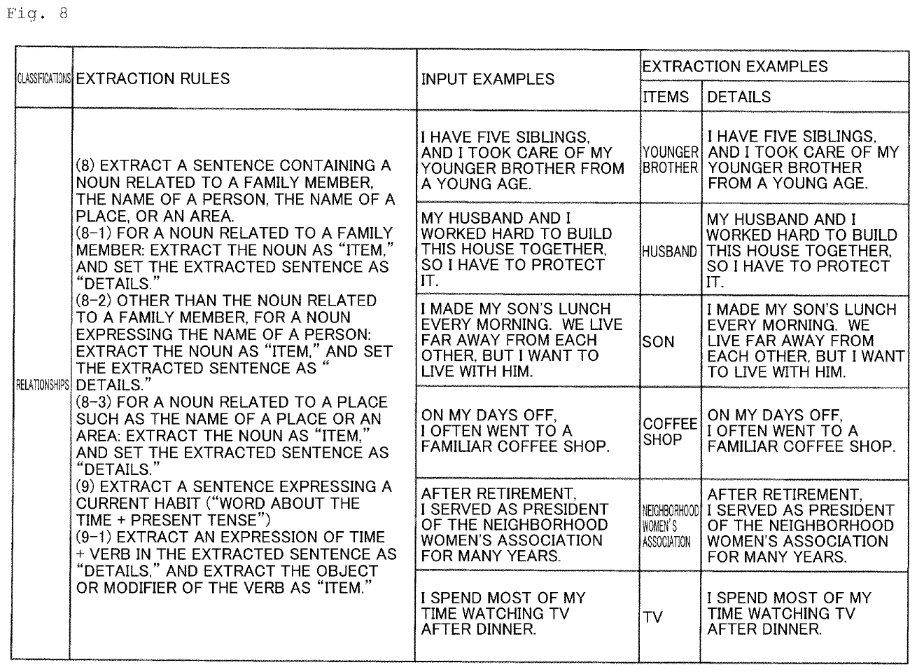

FIG. 8 is a diagram illustrating extraction rules for relationships and extraction examples for relationships. As for a relationship, there are a rule (8-1) and a rule (8-2) based on a rule (8) and a rule (9-1) based on a rule (9).

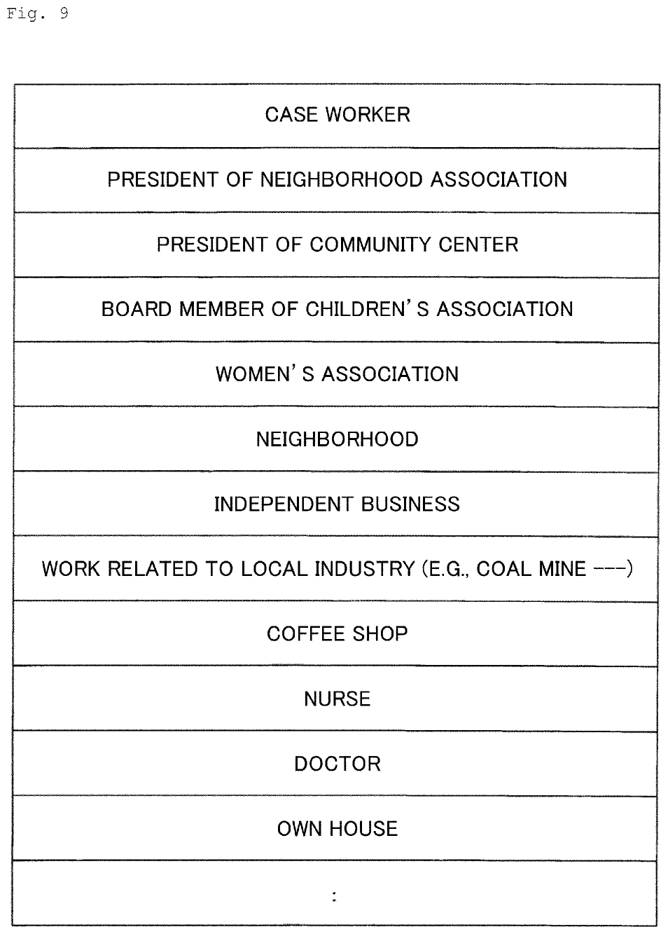

The rule (8) is an extraction rule for extracting a sentence including nouns related to a family, the name of a person, the name of a place, and an area. Note that the noun related to an area may be determined by referring to, for example, the dictionary information shown in FIG. 9. The dictionary information shown in FIG. 9 includes a set of nouns related to areas. Not only the nouns that exactly match the dictionary information, but also nouns containing such nouns or nouns containing such nouns at the end may be extracted as the corresponding nouns.

The rule (8-1) is an extraction rule for extracting the noun as “item” when the noun is related to a family, and setting the sentence extracted based on the rule (8) as “details.”

The rule (9) is an extraction rule for extracting a sentence expressing a current habit. For example, a sentence having a format such as “a word about the time+present tense” may be extracted as said sentence.

The rule (9-1) is an extraction rule for extracting an expression of time+verb as “details” in the sentence extracted on the basis of the rule (9), and extracting an object or modifier of the verb as “item.”

In FIG. 8, the meanings of the respective columns for “input example” and “extraction example” are the same as those in FIG. 4.

The extraction unit 11 records the person-in-question information (“item,” “details”) extracted for each classification from the recorded information, in the person-in-question information storage unit 122.

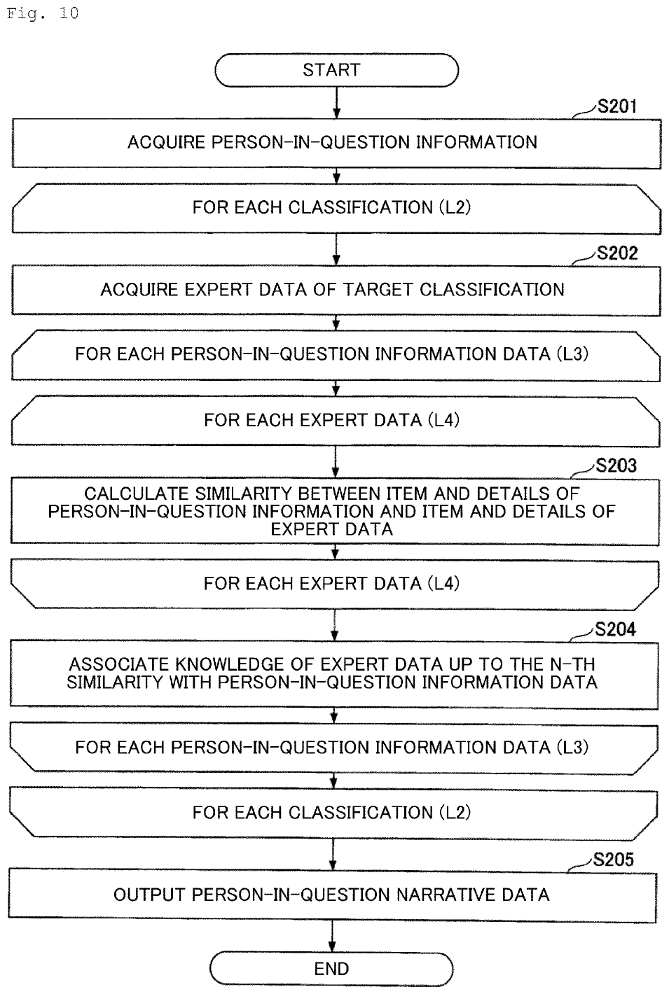

FIG. 10 is a flowchart for describing an example of a processing procedure executed by the acquisition unit 12 according to the first embodiment. The processing procedure of FIG. 10 may be executed following FIG. 3 or asynchronously with FIG. 3.

In step S201, the acquisition unit 12 acquires data on the person-in-question information of the target person (referred to as “person-in-question information data” hereinafter) from the person-in-question information storage unit 122. The person-in-question information data is a record of a unit discriminated for each combination of a classification, an item and details of the person-in-question information in the person-in-question information storage unit 122. Then, the acquisition unit 12 executes step S202 and loop processing L2 including loop processing L3, for each classification of the person-in-question information (“basic information,” “ability/experience,” “hobby/preference,” and “relationship”). The classification being processed in the loop processing L2 is hereinafter referred to as “target classification.”

In step S202, the acquisition unit 12 acquires expert data corresponding to the target classification from the expert DB123.

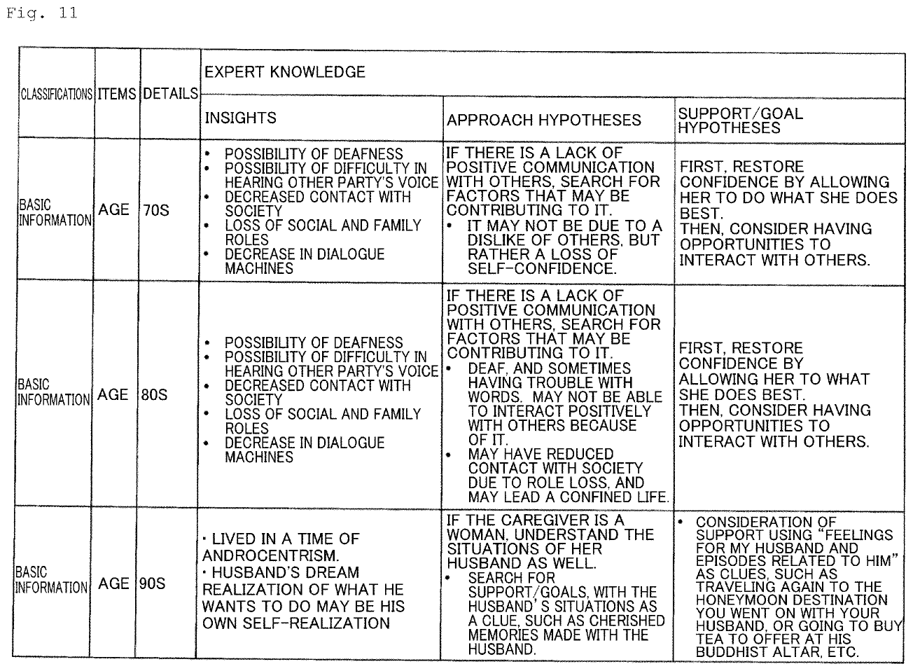

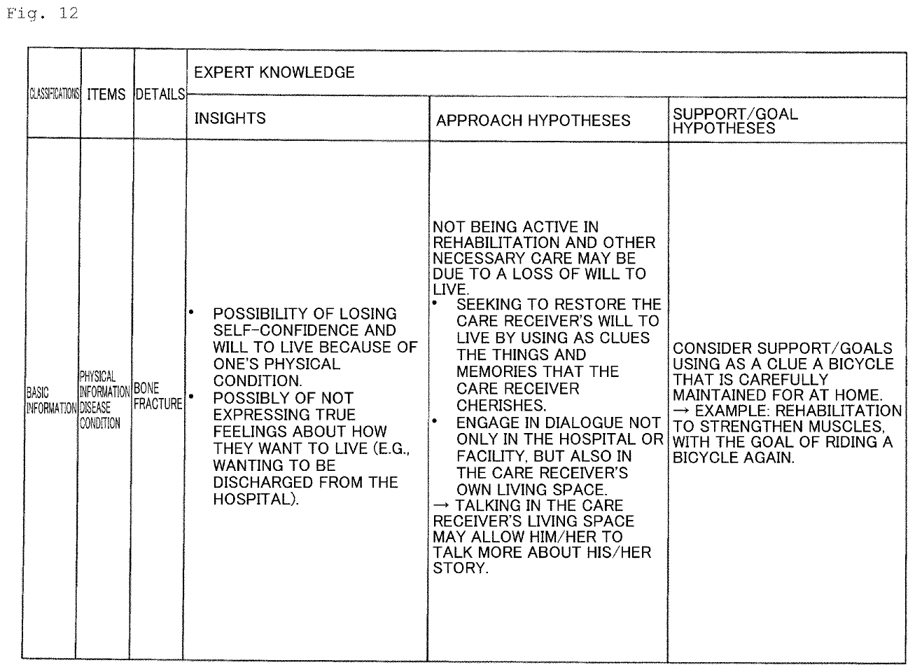

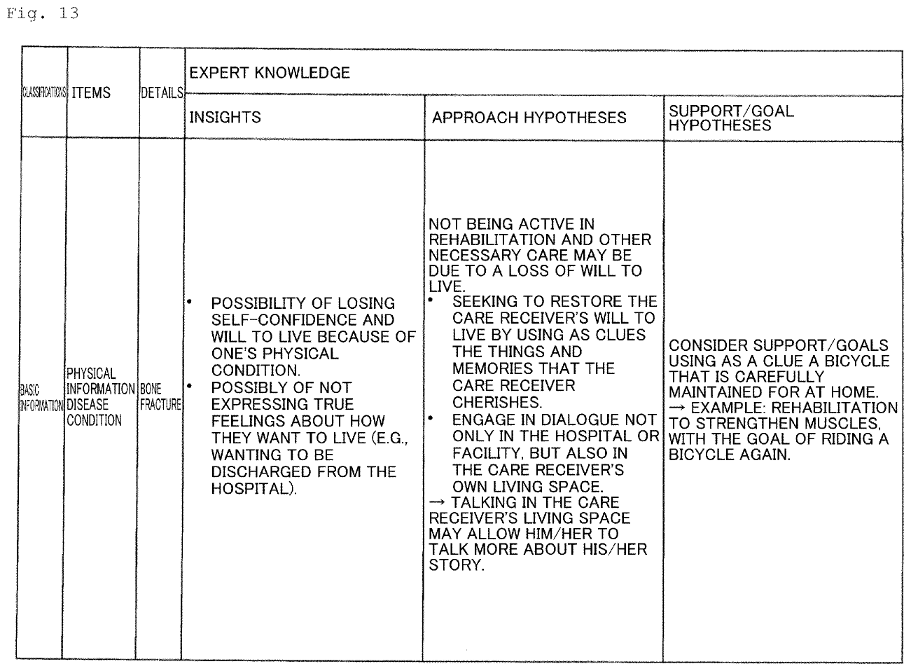

FIGS. 11 to 17 are each a diagram illustrating an example of a configuration of the expert DB123. As shown in FIGS. 11 to 17, in the Expert DB123, for each classification, there is data associated with knowledge (referred to as “expert data” hereinafter) are registered in advance with respect to information indicating a feature of a person, as distinguished by a combination of items and details.

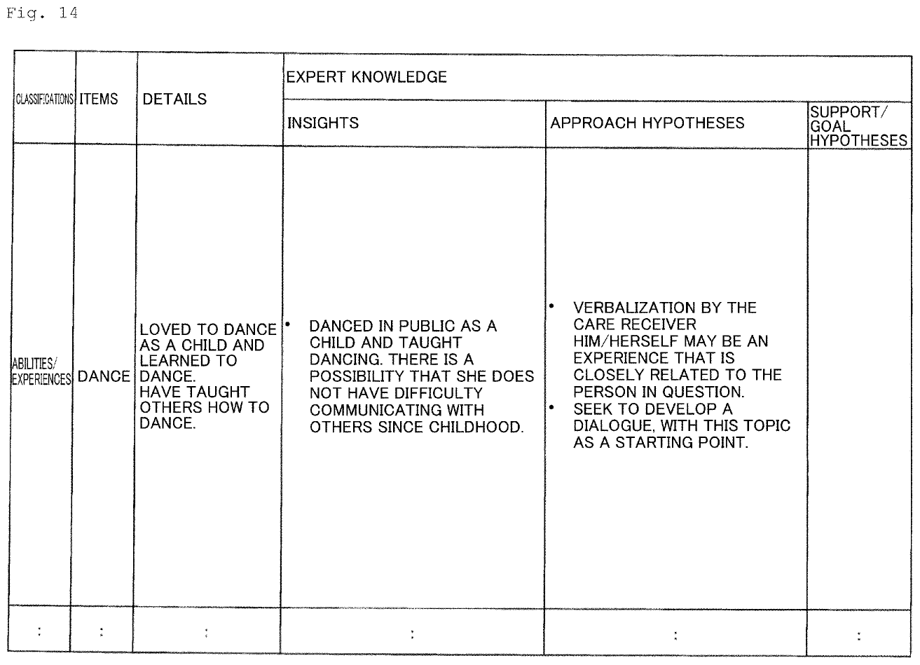

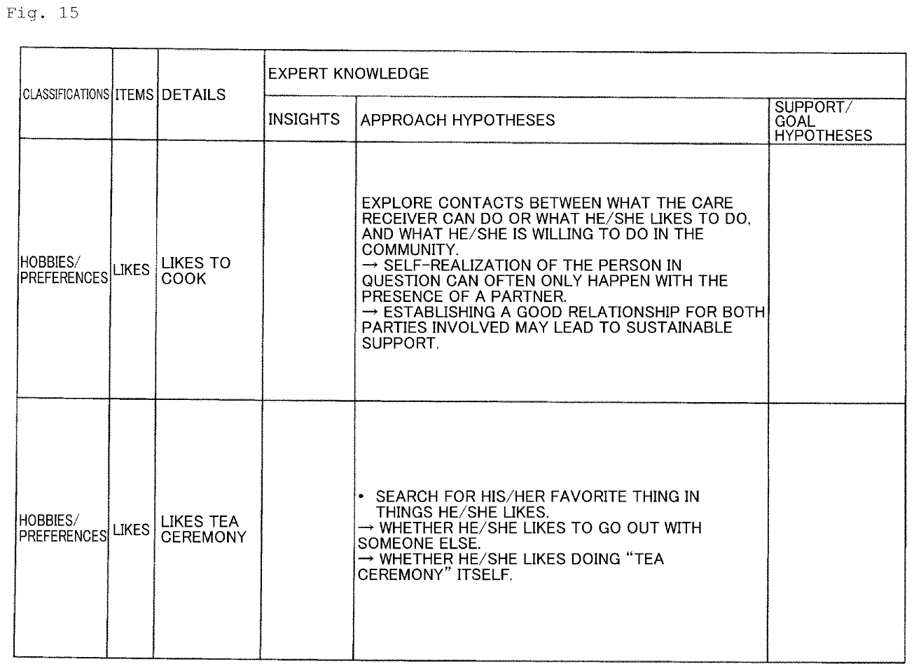

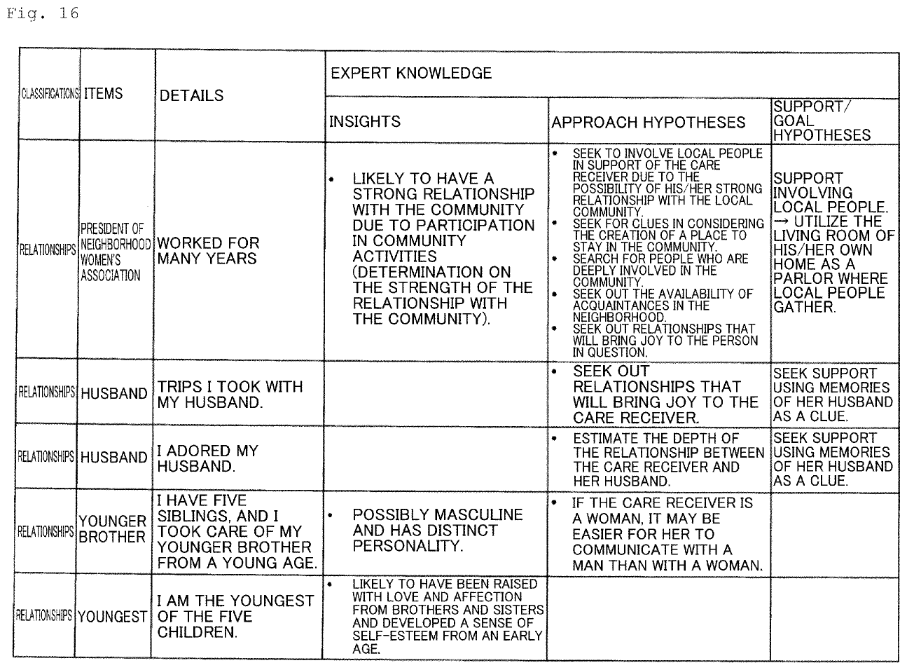

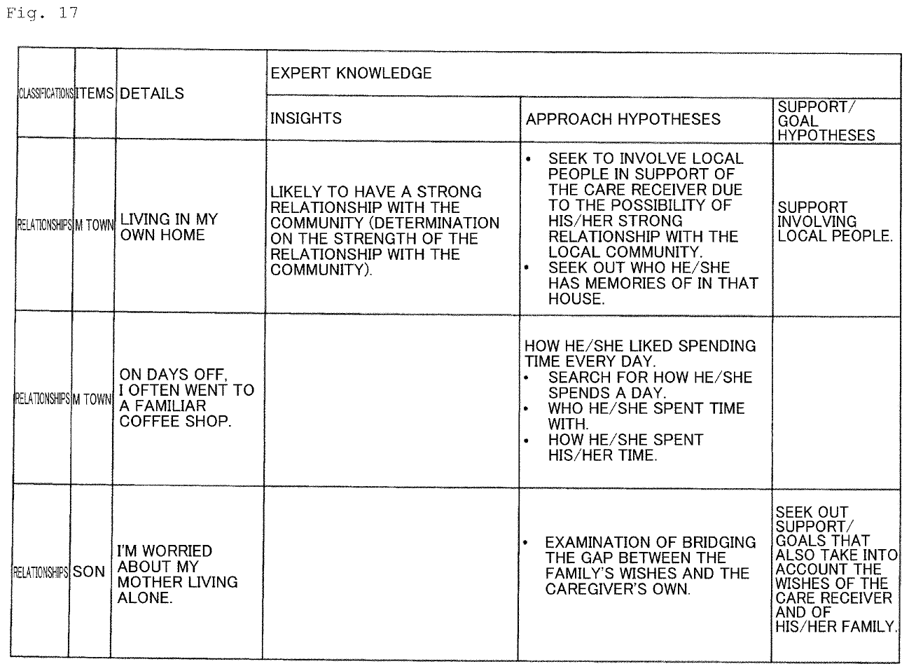

FIGS. 11 to 13 each show an expert data group related to the basic information. FIG. 14 shows an expert data group related to abilities/experiences. FIG. 15 shows an expert data group related to hobbies/preferences. FIGS. 16 and 17 each show an expert data group related to relationships.

The knowledge means information indicating that a specific person such as an expert uses his/her own knowledge to decipher the character of the care receiver and the feelings that the care receiver cannot put in words, for each pattern of typical person-in-question information (combination of an item and details). The knowledge is utilized, for example, for the caregiver to understand the care receiver him/herself, to approach the care receiver, and to derive hypotheses for the support/goals for the care receiver.

As shown in FIGS. 11 to 17, the knowledge according to the present embodiment includes (1) insights, (2) approach hypotheses, and (3) support/goal hypotheses. The insight here are defined as the character and trends in thinking, behaviors, physical conditions and the like that can be read from the combined information of categories, items, and details. The approach hypotheses are defined as a human relationship to be taken into consideration when considering the support/goals for the care receiver, the depth of the relationship with the area, social involvement, and the like.

In step S202, among the expert data groups registered in the expert DB123, the expert data group belonging to the target classification (hereinafter referred to as the “target expert data group”) is acquired.

Then, the acquisition unit 12 executes loop processing L3 including loop processing L4 and step S204, for each piece of person-in-question information data extracted with respect to the target classification. The data being processed in the loop processing L3 is hereinafter referred to as “target person-in-question information data.”

In loop processing L4, the acquisition unit 12 executes step S203 for each piece of expert data included in the target expert data groups. The expert data being processed in the loop processing L4 is hereinafter referred to as “target expert data.”

In step S203, the acquisition unit 12 calculates similarity between items and details of the target person-in-question information data and items and details of the target expert data. Here, the similarity between the items and details of one side and the items and details of the other may be the similarity between a character string group obtained by connecting the items and details of one side and a character string group obtained by connecting the items and details of the other. Alternatively, the similarity may be calculated for each item and for each detail, and the average or weighted average of the two similarities may be calculated. In the case of the weighted average, the ratio of the number of characters between the item and the detail may be used as the weight. The similarity between the character strings may be calculated by using any known method.

When the loop processing L4 is finished, the target person-in-question information data is in a state where similarity with each piece of expert data included in the target expert data group is calculated. For example, when m pieces of expert data are included in the target expert data group, m similarities are calculated.

Following the loop processing L4, the acquisition unit 12 associates knowledge of the expert data up to the n-th similarity with the target person-in-question information data (S204). That is, based on the similarity between items and details of the target person-in-question information data and items and details of each piece of expert data, expert data associated with the target person-in-question information data is selected. However, when a threshold is provided for the similarity and the similarity equal to or greater than the threshold is not calculated, the expert data associated with the target person-in-question information data may not be selected. In this case, expert data for the target person-in-question information data becomes insufficient.

When the loop processing L3 is executed for all the person-in-question information data and the loop processing L2 is executed for all the classifications, the acquisition unit 12 outputs, for example, a set of data in which knowledge is associated with the person-in-question information data of the target person (referred to as “person-in-question narrative table” hereinafter). In this case, the person-in-question narrative table is recorded in the narrative DB124 in association with identification information (name, ID, or the like) of the target person. In addition, the person-in-question narrative table may be displayed on a display device connected to the information processing apparatus 10 or a terminal connected to the information processing apparatus 10 via a network.

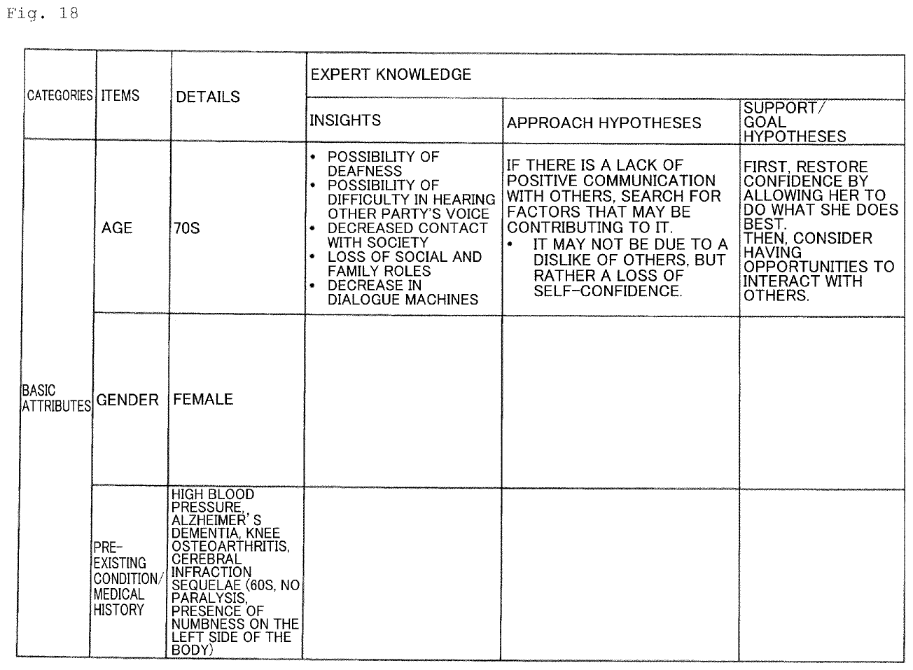

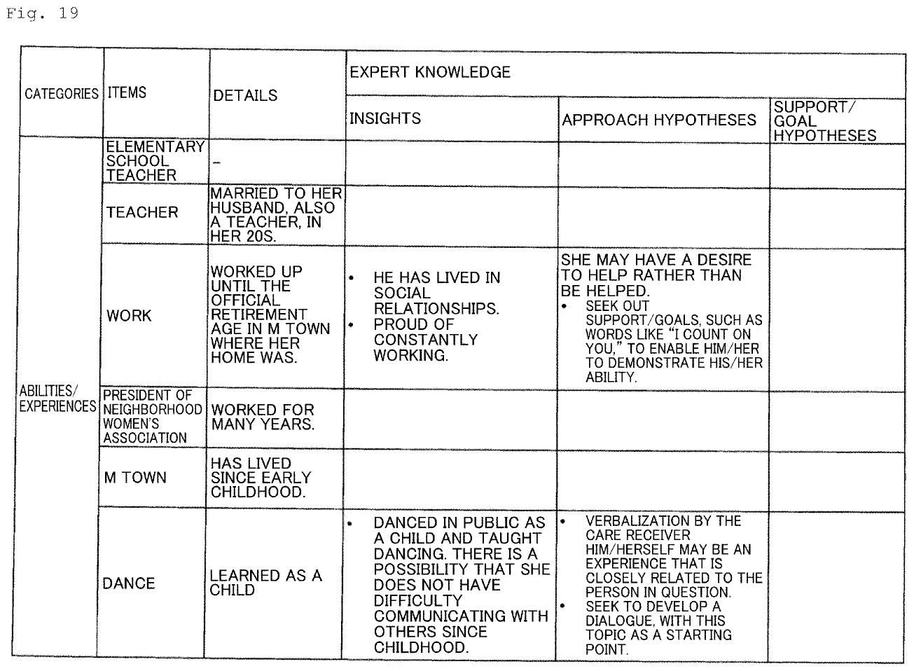

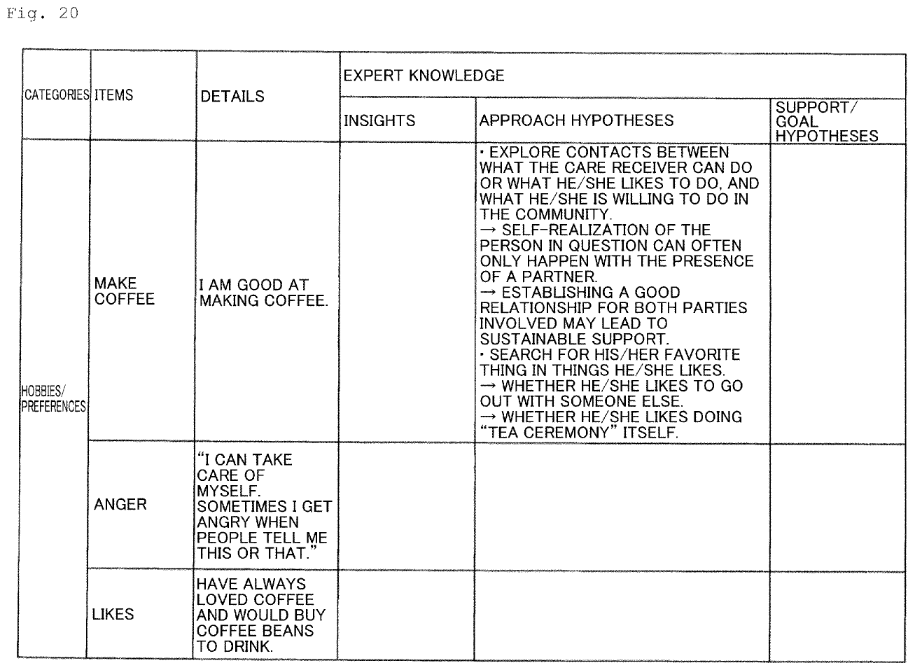

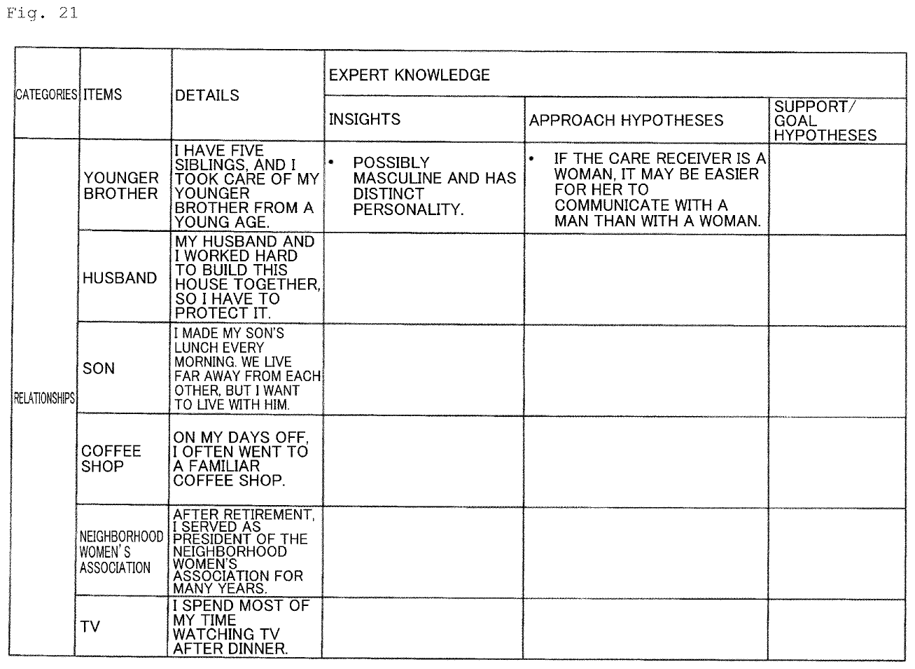

FIGS. 18 to 21 are each a diagram illustrating an example of a configuration of the person-in-question narrative table. For convenience, FIGS. 18 to 21 each illustrate the person-in-question narrative table for each classification, but the person-in-question narrative table is a person-in-question narrative table for one target person (individual) as a whole. That is, the person-in-question narrative table is generated for each individual. FIG. 18 illustrates a data group related to the basic information in the person-in-question narrative table. FIG. 19 illustrates a data group related to ability/experience in the person-in-question narrative table. FIG. 20 illustrates a data group related to hobby/preference in the person-in-question narrative table. FIG. 21 illustrates a data group related to a relationship in the person-in-question narrative table.

As shown in FIGS. 18 to 21, the person-in-question narrative table is data in which the knowledge of the expert is associated with the person-in-question information. When the processing procedures after FIG. 3 are periodically executed at intervals of cycle T1, the person-in-question narrative table that is output in step S205 includes knowledge in the latest cycle T1.

As described above, according to the first embodiment, it is possible to obtain implicit information based on the knowledge of the expert by using the daily explicit recorded information about a certain person (target person). Therefore, understanding of the implicit information about a certain person can be supported. As a result, the target person can expect care that is attuned to his/her personality and feelings by a care manager and other care professionals, community life support coordinators, social workers, and the like.

Although the present embodiment has described an example in which the candidate of the target person is a care receiver, the candidate of the target person may be, for example, a child, a person who receives assistance from another person, or the like. The expert in this case is an expert on said assistance.

A second embodiment will be described next. Differences from the first embodiment will be described in the second embodiment. Points not specifically mentioned in the second embodiment may be the same as those of the first embodiment.

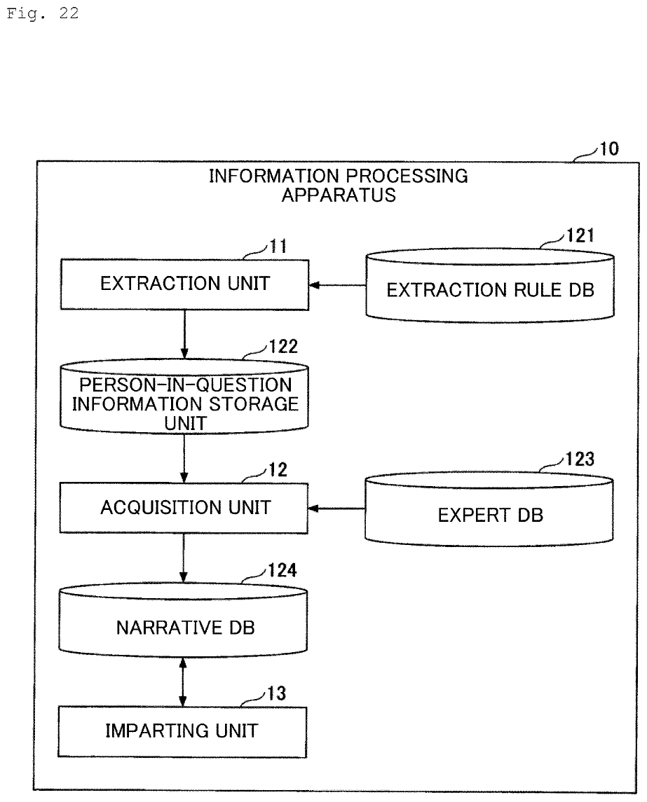

FIG. 22 is a diagram illustrating an example of a functional configuration of the information processing apparatus 10 according to the second embodiment. In FIG. 22, the same portions as those in FIG. 2 are designated by the same reference numerals, and the description thereof will be omitted accordingly.

In FIG. 22, the information processing apparatus 10 further includes an imparting unit 13. The imparting unit 13 is realized by causing the CPU 104 to execute one or more programs installed in the image processing apparatus 10.

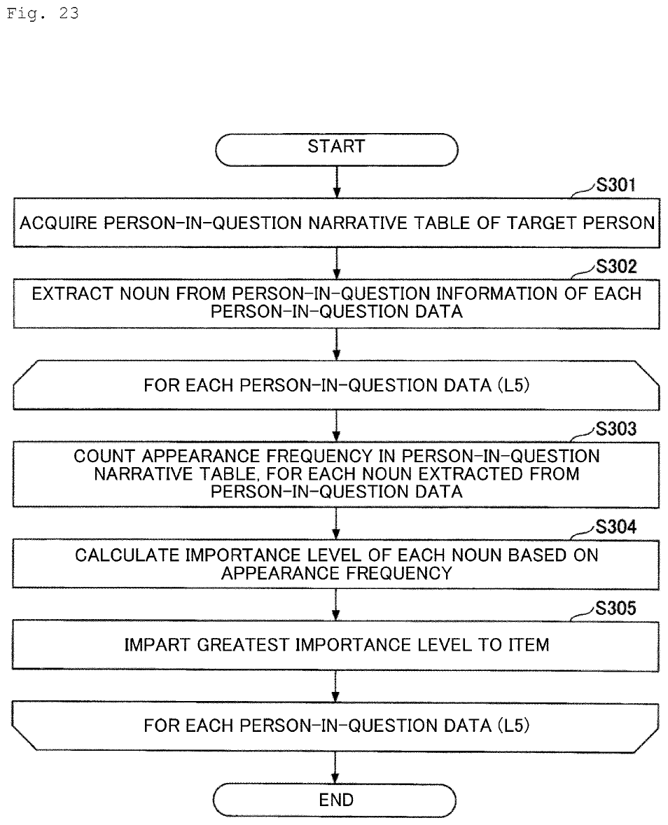

FIG. 23 is a flowchart for describing an example of a processing procedure executed by the imparting unit 13 according to the second embodiment. The processing procedure shown in FIG. 23 is executed after the processing procedure shown in FIG. 10 is executed for the target person.

In step S301, the imparting unit 13 acquires the latest person-in-question narrative table of the target person (FIGS. 18 to 21) from the narrative DB124. The latest person-in-question narrative table refers to a person-in-question narrative table generated for the target person in the latest cycle T1.

Subsequently, the imparting unit 13 extracts nouns from the person-in-question information (items and details) in the person-in-question data, for each unit that separates the person-in-question narrative table for each combination of items and details (hereinafter referred to as “person-in-question data”) (S302). Then, the imparting unit 13 executes loop processing L5 for each person-in-question data. The loop processing L5 includes steps S303 to S305. Hereinafter, the person-in-question data being processed in the loop processing L5 is hereinafter referred to as “target person-in-question data.”

For each noun extracted in step S302 with respect to the target person-in-question data, in step S303, the imparting unit 13 counts appearance frequencies (number of occurrences) in all person information (i.e., person-in-question information of all person-in-question data (items and details) in the person-in-question narrative table. In this case, the synonym of each noun may also be counted as the appearance frequency of the noun. For example, the appearance frequency of “elementary school teacher” or “teacher” may be added to the appearance frequency. Further, for the appearance frequency of “when little,” “early childhood” and “as a child” may be added to the appearance frequency of “when little.” Note that the synonym of each noun may be determined by using, for example, a synonym dictionary which is disclosed.

Then, for each noun in the target person-in-question data, the imparting unit 13 calculates a weight based on the counted appearance frequency for said noun (hereinafter referred to as “importance level”) (S304). For example, the importance level of a certain noun may be calculated as a ratio of the appearance frequency of the certain noun to the total number of the nouns of the person-in-question information (items and details) of all the person-in-question data in the person-in-question narrative table. For example, let it be assumed that the total number is 42, that the appearance frequency of “coffee” is 2, and that the appearance frequency of “early childhood” is 3. In this case, TF coffee and TF early childhood, which are the importance level of each of “coffee” and “early childhood,” may be calculated as follows.

TF coffee=2/42=0.05

TF early childhood=3/42=0.07

Subsequently, the imparting unit 13 imparts the greatest importance level to the target person-in-question data out of the importance levels calculated for the respective nouns of the target person-in-question data, and writes back the target person-in-question data with the imparted importance level to the narrative DB124 (S305). Here, it is considered that the importance level indicates the importance level for the person-in-question. Therefore, by imparting an importance level to each piece of person-in-question data, a difference in importance level for each piece of person-in-question data can be given to the narrative DB124.

Although only the latest person-in-question narrative table is taken into consideration in the calculation of importance levels, importance levels may be calculated by using the person-in-question narrative tables of a target person for the past x times that are generated periodically. For example, the importance level of noun a may be calculated based on the following formula.

Importance level=Appearance frequency latest noun a/total number of latest nouns×log(total number of nouns in past×times/appearance frequency of noun a in past×times)

This formula is based on TF−IDF.

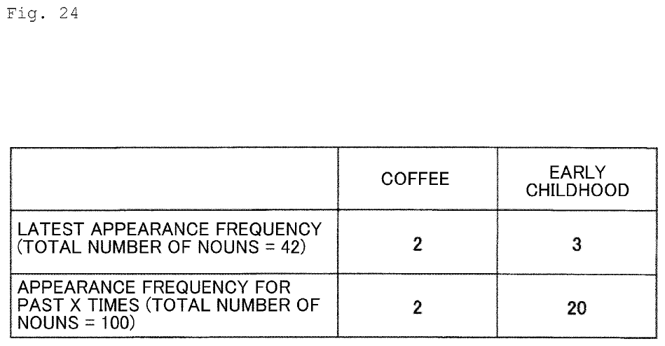

Specific examples of this case are now described. FIG. 24 is a diagram for explaining an example of calculating an importance level in consideration of a past person-in-question narrative table. FIG. 24 shows the appearance frequency of each of the nouns of “coffee” and “early childhood” in the latest person-in-question narrative table and the appearance frequency of the same in the person-in-question narrative tables of the past x times (including the latest one). In this case, based on the above formula, the importance levels of “coffee” and “early childhood” (TF coffee, TF early childhood) are calculated as follows.

TF coffee=2/42×log(100/2=0.19

TF early childhood=3/42×log(100/20)=0.11

However, the importance level of each noun may be calculated by other known weighting techniques.

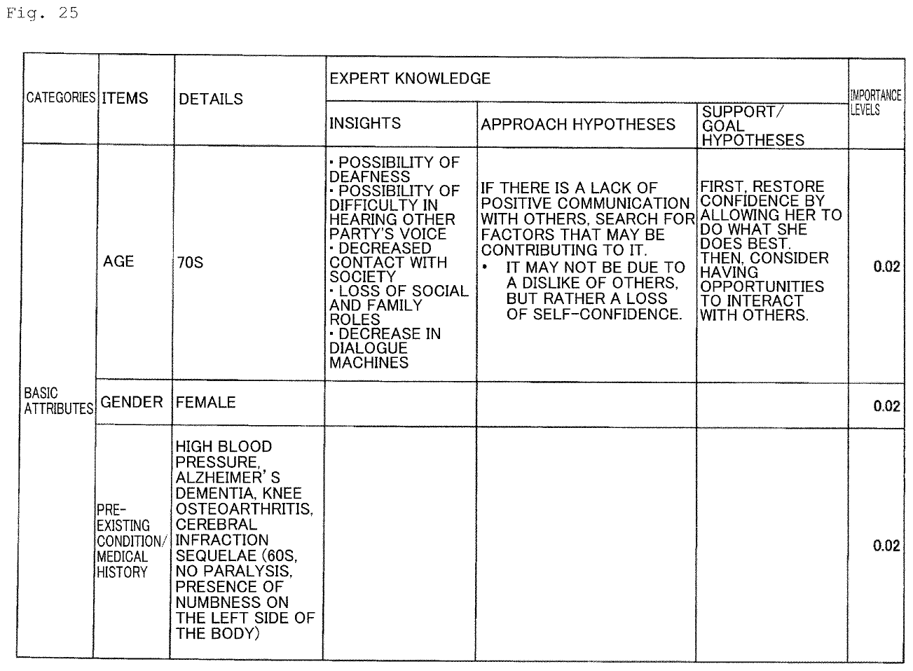

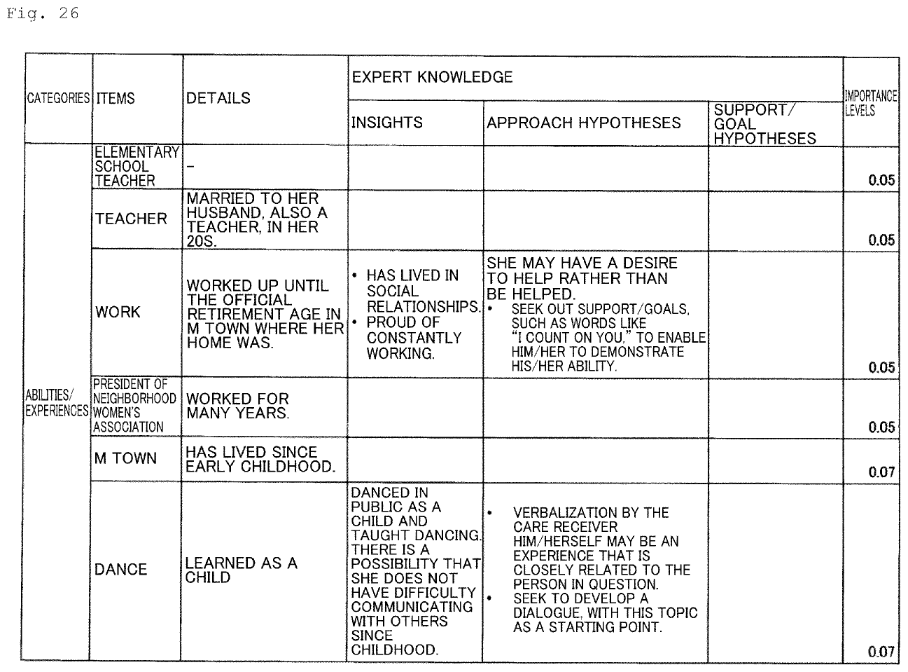

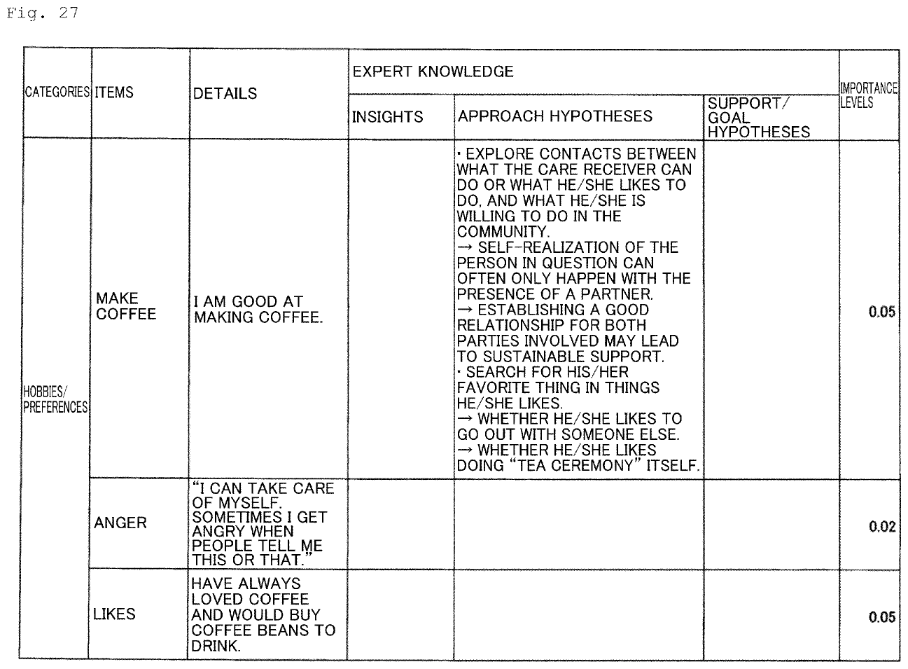

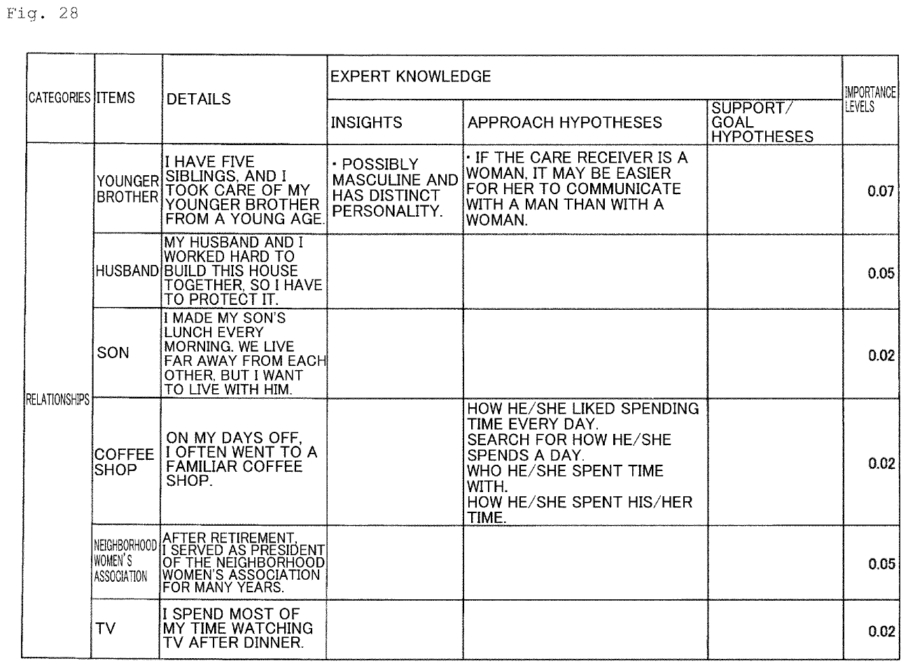

By executing the processing procedure shown in FIG. 23, the person-in-question native tables shown in FIGS. 18 to 21 are updated as shown in FIGS. 25 to 28.

The imparting unit 13 may output the person-in-question narrative tables to which the importance levels are imparted by a method such as display.

As described above, according to the second embodiment, an importance level is imparted to each piece of person-in-question data constituting the person-in-question narrative tables. Since the importance of each piece of person-in-question data is not uniform for the person in question, the information to be intensively considered can be presented when a professional sets a target/problem by performing weighting.

A third embodiment will be described next. Differences from the first embodiment will be described in the third embodiment. Points not specifically mentioned in the third embodiment may be the same as those of the first embodiment.

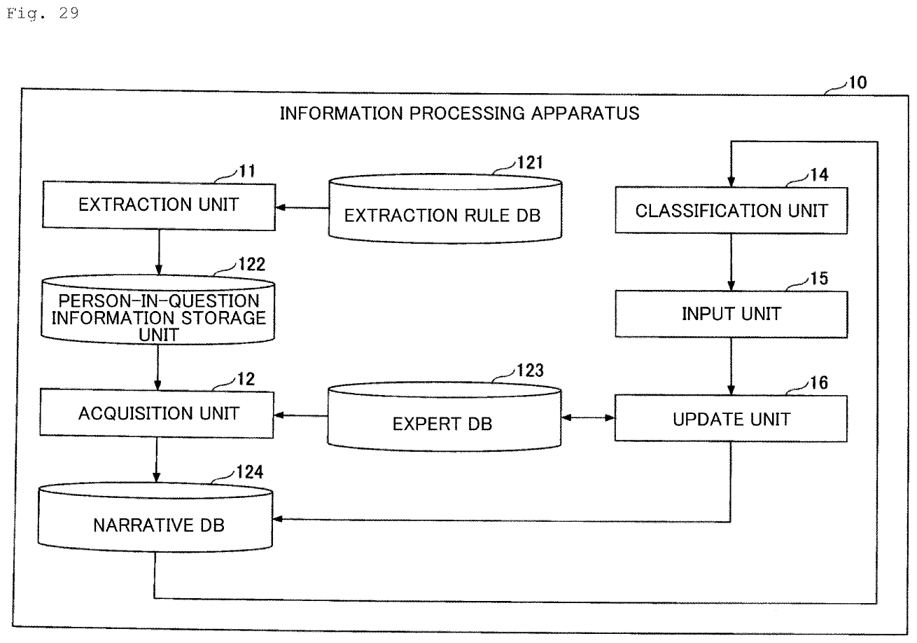

FIG. 29 is a diagram illustrating an example of a functional configuration of the information processing apparatus 10 according to the third embodiment. In FIG. 29, the same portions as those in FIG. 2 are designated by the same reference numerals, and the description thereof will be omitted accordingly.

In FIG. 29, the information processing apparatus 10 further includes a classification unit 14, an input unit 15, and an update unit 16. Each of the units is realized by processing of causing the CPU 104 to execute one or more programs installed in the information processing apparatus 10.

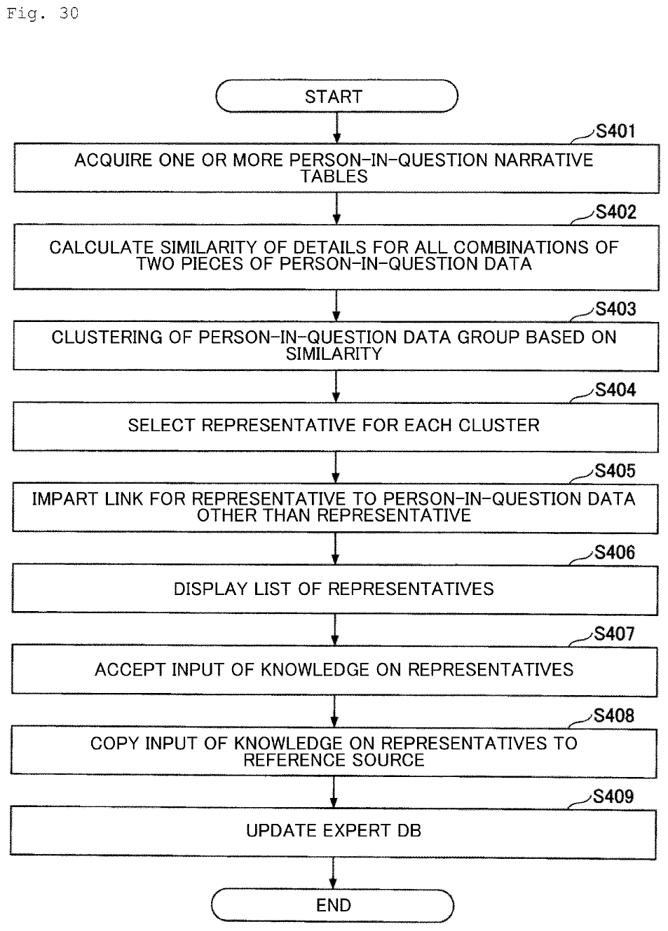

FIG. 30 is a flowchart for describing an example of a processing procedure executed by the information processing apparatus 10 according to the third embodiment.

In step S401, the classification unit 14 acquires from the narrative DB124, for example, a person-in-question narrative table of each of one or more care receivers designated by a user. Here, the person-in-question narrative table is a set of person-in-question data for each classification. Note that a target to be acquired may be a latest person-in-question narrative table or a plurality of past person-in-question narrative tables.

Subsequently, the classification unit 14 calculates the similarity of character strings included in the “details” for all combinations of two pieces of person-in-question data out of the set of all person-in-question data included in all the acquired person-in-question narrative tables (referred to as “all person-in-question data groups” hereinafter) (S402). The similarity between the character strings may be calculated by using a known method.

Subsequently, the classification unit 14 performs clustering on all person-in-question data groups on the basis of the similarity of each combination of the two pieces of person-in-question data (similarity), and classifies the all person-in-question data groups into a plurality of clusters (groups) (S403). The clustering may be performed based on a known method. Subsequently, the classification unit 14 selects, for each cluster, person-in-question data having the largest information amount included in “details” in the person-in-question data group belonging to the cluster, as representative data. That is, person-in-question data is selected one by one for each cluster. The information amount may be measured by the number of words or by the number of characters. Then, for each cluster, the classification unit 14 assigns (records) the ID of the representative data to the person-in-question data other than the representative data of the cluster (hereinafter referred to as “non-representative data”) (S405).

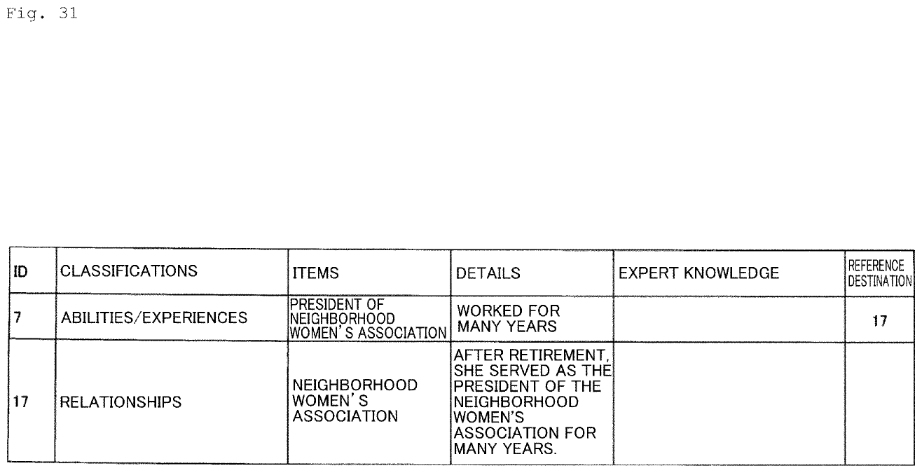

FIG. 31 is a diagram illustrating an example of assigning an ID of representative data to non-representative data. FIG. 31 illustrates an example of a cluster including two pieces of person-in-question data. In FIG. 31, the second person-in-question data of “details” has a larger amount of information of “details.” Therefore, the second person-in-question data is selected as the representative data, and the ID of the representative data is recorded as the value of “reference destination” in the first person-in-question data. That is, a link to the representative data is provided to the non-representative data. In the first embodiment, the ID of each piece of person-in-question data is omitted for convenience.

Subsequently, the input unit 15 displays a screen containing a list of representative data (referred to as “representative data list screen” hereinafter) on a user terminal (S406). The user terminal is a terminal used by the user (expert). On the representative data list screen, the column “expert knowledge” of each piece of representative data is made editable.

Then, the input unit 15 accepts input (new input or update (revision)) for “expert knowledge” of any of the representative data in the representative data list screen, and reflects said input to the representative data in the narrative DB124 (S407).

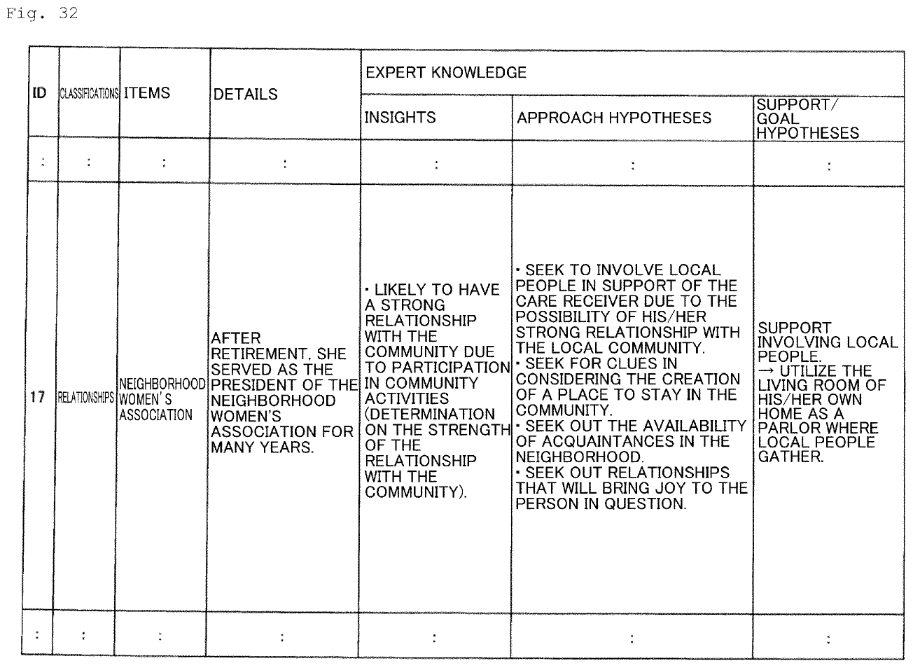

FIG. 32 is a diagram illustrating an example of accepting input for “expert knowledge” of the representative data. FIG. 32 illustrates an example in which a new expert's opinion is input for “insights,” “approach hypotheses,” and “support/goal hypotheses” of the “expert knowledge” for the person-in-question data selected as representative data in FIG. 31. The representative data to be input for “expert knowledge” out of one or more pieces of representative data included in the representative data list screen may be limited to the representative data in which “expert knowledge” is empty (i.e., “expert knowledge” is insufficient). In other words, only such representative data may be included in the representative data list screen. However, the existing contents may be edited (changed) for the representative data to which “expert knowledge” has already been input.

Alternatively, only representative data of clusters in which the number of pieces of data containing “expert knowledge” is less than a predetermined number (or the number of pieces of applicable data is 0), or representative data of clusters in which the ratio of the number of pieces of data containing “expert knowledge” to the total number of pieces of data in the person-in-question data group is less than a predetermined value, may be included in the representative data list screen.

If there exists data in the person-in-question data group belonging to the cluster that includes “expert knowledge” in the data other than the representative data, the “expert knowledge” in the person-in-question data group belonging to the cluster may be posted (may be merged with the contents of the “expert knowledge” column originally included in the representative data) in the “expert knowledge” column of the representative data. The representative data thus created may be displayed in a representative data list so that the “expert knowledge” column can be edited. Conversely, the representative data may not be displayed in the representative data list.

Subsequently, the update unit 16 copies the contents of “expert knowledge” of representative data input to “expert knowledge,” to “expert knowledge” of the non-representative data which is a reference source of the representative data in the narrative DB124 (S408). The non-representative data as the reference source of the representative data is non-representative data in which the ID of said representative data is recorded in “reference destination.”

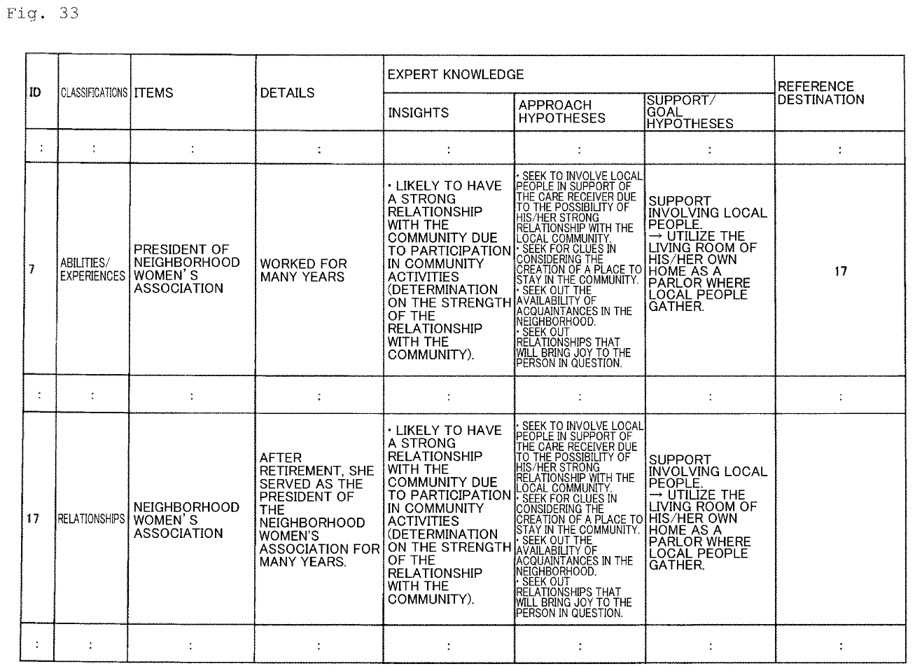

FIG. 33 is a diagram illustrating an example of copying “expert knowledge” to non-representative data. FIG. 33 illustrates an example in which input contents of the representative data of FIG. 31 for “expert knowledge” is copied to “expert knowledge” of the non-representative data shown in FIG. 31.

In this manner, the input of knowledge on one piece of person-in-question data by the expert is copied to one or more pieces of data similar to said person-in-question data.

Subsequently, for each cluster of representative data whose “expert knowledge” has been updated (referred to as “updated cluster” hereinafter), the update unit 16 adds, as new expert data to the expert DB123, the data including “classification,” “item,” and “expert knowledge” of each piece of person-in-question data in the updated cluster, and including the “details” with the highest information abstraction level among the “details” of the person-in-question data in the updated cluster (S409). That is, the result of the input by the expert is reflected also in the expert DB123. Here, the level of information abstraction may be evaluated by the number of words or the number of characters. For example, “detail” having the smallest number of words may be determined as “detail” having the highest information abstraction level.

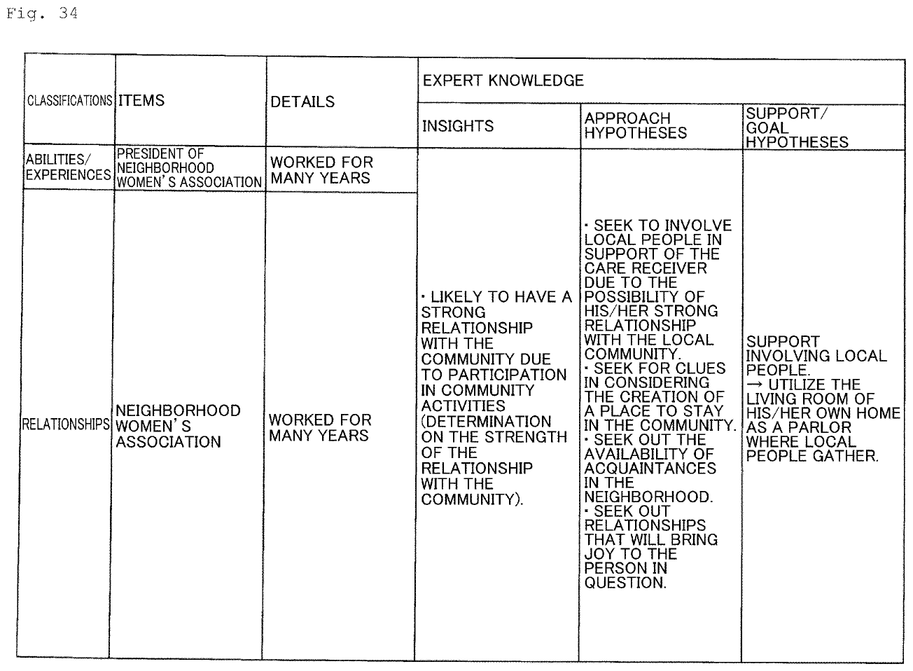

For example, in the cluster shown in FIG. 31, the level of abstraction for “details” of the first person-in-question data is the highest. Therefore, when “expert knowledge” shown in FIG. 33 is recorded for each piece of person-in-question data of the cluster, the expert data shown in FIG. 34 is added to the expert DB123.

FIG. 34 is a diagram illustrating a first example of the expert data added to the expert DB123. In FIG. 34, “classification” and “item” of the first expert data are “classification” and “item” of the first person-in-question data in FIG. 31, and “classification” and “item” of the second expert data are “classification” and “item” of the second person-in-question data shown in FIG. 31. However, “details” of each piece of expert data in FIG. 34 are “details” of the first person-in-question data in the second person-in-question data shown in FIG. 31. In this manner, “details” having the highest abstraction level is to be registered in the expert data because it is expected that the matching rate is improved in the collation between the person-in-question information and the expert data by the acquisition unit 12. In FIG. 34, since “expert knowledge” of each piece of expert data is common, “expert knowledge” of each piece of expert data is integrally expressed in FIG. 34 for convenience.

However, for each “classification” and “item” of each person-in-question data in the updated cluster, expert data for each variation of all “details” in the updated cluster may be added to the expert DB123.

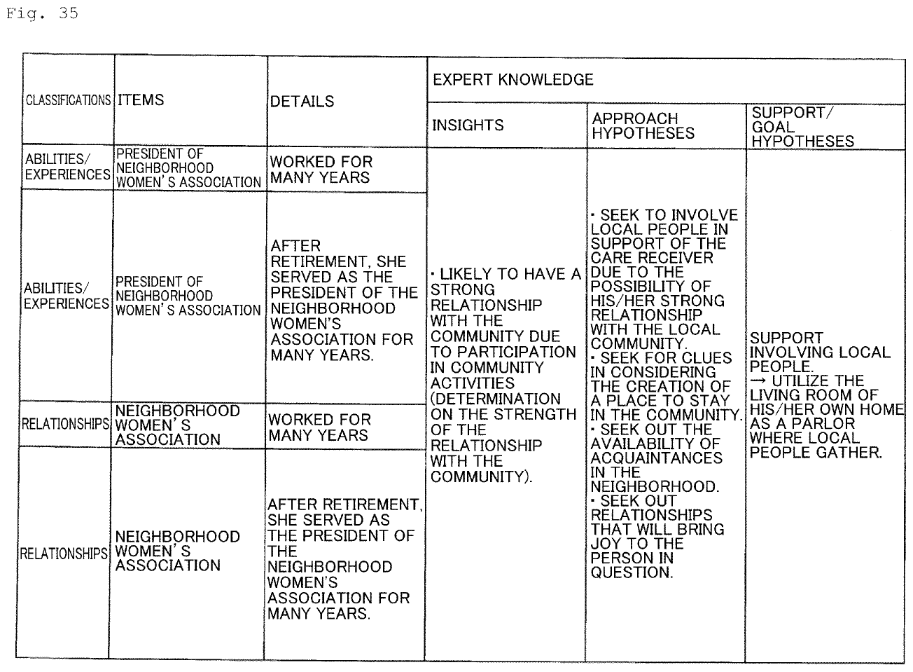

In this case, the expert data added to the expert DB123 with respect to the cluster shown in FIG. 31 is as shown in FIG. 35.

FIG. 35 is a diagram illustrating a second example of the expert data added to the expert DB123. In FIG. 35, the first expert data includes “classification,” “item,” and “details” of the first person-in-question data shown in FIG. 31. The second expert data includes “classification” and “item” of the first person-in-question data in FIG. 31 and “details” of the second person-in-question data. The third expert data includes “classification” and “item” of the second person-in-question data in FIG. 31, and “details” of the first person-in-question data. The fourth expert data includes “classification,” ““item,” and “details” of the second person-in-question data shown in FIG. 31.

Note that the third embodiment and the second embodiment may be combined.

As described above, according to the third embodiment, the knowledge of an expert can be efficiently acquired.

Although embodiments of the present invention have been described above in detail, the present invention is not limited to the specific embodiments described above, and various modifications and changes can be made within the concept of the present invention described in the claims.

### REFERENCE SIGNS LIST

10 Information processing apparatus
11 Extraction unit
12 Acquisition unit
13 Imparting unit
14 Classification unit
Input unit
16 Update unit
100 Drive device
101 Recoding medium
102 Auxiliary storage device
103 Memory device
104 CPU
105 Interface device
121 Rule DB
122 Person-in-question information storage unit
123 Expert DB
124 Narrative DB
B Bus

1. An information processing apparatus, comprising:
a memory; and
a processor coupled to the memory and configured to:
classify a plurality of pieces of data including pieces of personal feature information, each of which is a piece of information indicating a feature of an individual, into groups on the basis of similarity of the pieces of information indicating the features;
present one piece of data representing data included in the groups;
receive input of information that implicitly indicates a feature indicated by the personal feature information included in the data presented; and
record the data whose input of the information is received, in a storage unit, in association with the information received.

2. The information processing apparatus according to claim 1, wherein the information received is associated with data classified into the same group as the data whose input of the information is received.

3. An information processing method, comprising:
classifying a plurality of pieces of data including pieces of personal feature information, each of which is a piece of information indicating a feature of an individual, into groups on the basis of similarity of the pieces of information indicating the features;
presenting one piece of data representing data included in the groups;
receiving input of information that implicitly indicates a feature indicated by the personal feature information included in the data presented; and
recording the data whose input of the information is received, in a storage unit, in association with the information received.

4. A non-transitory computer-readable recording medium storing a program that causes a computer to function as the information processing apparatus according to claim 1.
